(19) United States
(12) Patent Application Publication
(10) Pub. No.: US 2026/0131130 A1
(43) Pub. Date: May 14, 2026

(54) FIXING-LOCKING STRUCTURE, MEDICAL CATHETER AND BLOOD PUMP

(71) Applicant: FORQALY MEDICAL (SHANGHAI) CO., LTD., Shanghai (CN)

(72) Inventors: Zhirong TANG, Shanghai (CN); Zhiyong LIU, Shanghai (CN); Lingyan XU, Shanghai (CN)

(73) Assignee: FORQALY MEDICAL (SHANGHAI) CO., LTD., Shanghai (CN)

(21) Appl. No.: 18/706,008

(22) PCT Filed: Nov. 3, 2022
(86) PCT No.: PCT/CN2022/129601

§ 371 Date: Apr. 30, 2024

(30) Foreign Application Priority Data
Nov. 3, 2021 (CN) 202111291103.4

(51) Int. Cl. A61M 60/414; A61M 60/13; A61M 60/216; A61M 60/405

(52) U.S. Cl. CPC A61M 60/414; A61M 60/13; A61M 60/216; A61M 60/405; A61M 2205/0216

(57) ABSTRACT

The present application provides a fixing-locking structure, including an inner cylinder body, a chuck and an outer cylinder body that sleeve a catheter. A first end of the outer cylinder body is provided with a first chamber to accommodate the chuck. A first end of the inner cylinder body is connected to the first end of the outer cylinder body. One end of the chuck abuts against an inner wall of the first end of the inner cylinder body, and the other end of the chuck abuts against an inner wall of the outer cylinder body corresponding to the first chamber. A clamping part is arranged at each of two ends of the chuck, and a compressing-fitting part is formed at a position where each of the inner cylinder body and the outer cylinder body abuts against the clamping part. The compressing-fitting part compresses the clamping part to contract inwards.

### CROSS-REFERENCE TO RELATED APPLICATIONS

This application is a National Stage of International Application No. PCT/CN2022/129601, filed on Nov. 3, 2022, which claims priority to Chinese Patent Application No. 202111291103.4, filed on Nov. 3, 2021, both of which are hereby incorporated by reference in their entireties.

### TECHNICAL FIELD

The present application relates to the technical field of medical apparatus, and in particular to a fixing-locking structure, a medical catheter and a blood pump.

### BACKGROUND

A route of a percutaneous catheter pump used in the clinical practice is to extract blood from the ventricle, flow through a guiding catheter crossing the valve and enter the artery from a flow outlet. This working principle determines that a suction inlet and the flow outlet need to be distributed on two sides of the valve. When a product is implanted, under the guidance of Digital Subtraction Angiography (DSA), the suction inlet and the flow outlet can be accurately distributed on two sides of the valve. After the product is in place, a suture system needs to be fixedly connected to a catheter through a locking structure, and then an entire catheter body needs to be fixed to a human body through the suture system. On one hand, if the locking between the locking structure and the catheter body are not reliable, during the process of assisting blood circulation, a force on the catheter will be displaced, which will change the arrangement of the suction inlet and the flow outlet that must be distributed on two sides of the valve, thereby causing the product to lose its clinical function; on the other hand, if the deformation of a sheath catheter at a locking position of the catheter is uncontrollable, it will cause the following effects on an internal structure of the sheath catheter: firstly, the unexpected structural changes such as catheter collapse may occur during locking; secondly, after locking, the minimum inner diameter of the sheath catheter decreases; during the clinical practice of a percutaneous intervention type blood pump, the catheter at least includes an internal mechanical transmission shaft and an external sheath catheter; if the deformation of the sheath catheter is uncontrollable, it will cause the malfunction of the mechanical transmission shaft inside the catheter, and in a severe case, it may cause the malfunction of the percutaneous catheter pump and the surgical failure. In order to detect the displacement of the catheter body in time, in similar products abroad, a very complex internal displacement alarm system has been developed, but the main function of the alarm system is only to remind the doctor to remedy the unexpected displacement of the catheter, and the best practice is to provide a reliable locking force to prevent the occurrence of displacement.

Therefore, the technical solution in the related art needs to be improved and enhanced.

### SUMMARY

The technical problem to be solved in the present application is to provide a fixing-locking structure, a medical catheter and a blood pump, which are fixed and locked outside a sheath catheter of a catheter through a chuck, and the chuck is provided with a slot, so as to ensure the fixing-locking effect and make the deformation of the sheath catheter controllable.

In order to solve the above-mentioned technical problem, a technical solution of the present application provides a fixing-locking structure including an inner cylinder body, a chuck and an outer cylinder body, and the inner cylinder body, the chuck and the outer cylinder body sleeve a catheter. The chuck is in a cylindrical shape. A first end of the outer cylinder body is provided with a first chamber to accommodate the chuck, a first end of the inner cylinder body is connected to the first end of the outer cylinder body, one end of the chuck abuts against an inner wall of the first end of the inner cylinder body, and the other end of the chuck abuts against an inner wall of the outer cylinder body corresponding to the first chamber. The end of the chuck abutting against the inner cylinder body is provided with a first clamping part, the inner wall of the first end of the inner cylinder body is provided with a first compressing-fitting part that abuts against the first clamping part, the end of the chuck abutting against the outer cylinder body is provided with a second clamping part, and the inner wall of the outer cylinder body corresponding to the first chamber is provided with a second compressing-fitting part that abuts against the second clamping part. When the first end of the inner cylinder body is connected to the first end of the outer cylinder body, the first compressing-fitting part compresses the first clamping part to contract the first clamping part inwards, and the second compressing-fitting part compresses the second clamping part to contract inwards.

Further, the chuck is formed as an elastic body.

Further, the first clamping part is provided with a first slot in a circumferential direction, the second clamping part is provided with a second slot in the circumferential direction, both of the first slot and the second slot are formed on an outer peripheral face of the chuck and extend in an axial direction of the chuck, and the first slot and the second slot are staggered with each other in the circumferential direction of the chuck.

Further, at least two first slots are formed on the chuck and evenly spaced apart from one another, and at least two second slots are formed on the chuck and evenly spaced apart from one another.

Further, each of the first clamping part, the second clamping part, the first compressing-fitting part and the second compressing-fitting part is in a shape of a frustum of a cone, a top angle of the cone of the first clamping part is smaller than a top angle of the cone of the first compressing-fitting part, and a top angle of the cone of the second clamping part is smaller than a top angle of the cone of the second compressing-fitting part.

Further, the first end of the inner cylinder body is connected to the first end of the outer cylinder body through a threaded connection structure, the threaded connection structure includes an external thread arranged on an outer wall of the first end of the inner cylinder body and an internal thread arranged on an inner wall of the first end of the outer cylinder body, and the external thread matches with the internal thread.

Further, the first end of the inner cylinder body is connected to the first end of the outer cylinder body through a snapping connection structure, the snapping connection structure includes a positioning pin arranged on an outer wall of the first end of the inner cylinder body and a ring groove formed on an inner wall of the first end of the outer cylinder body, the ring groove is formed in a circumferential direction of the inner wall of the outer cylinder body, a guiding groove is formed in an axial direction of the inner wall of the first end of the outer cylinder body; the guiding groove communicates with the ring groove, the positioning pin passes through the guiding groove in the axial direction, slidably moves into the ring groove and rotates to be fixed into the ring groove, and the inner cylinder body is fixed into the ring groove of the outer cylinder body through the positioning pin.

Further, a minimum inner diameter of a sheath catheter of the catheter after being compressed, locked and fixed by the chuck is estimated by a following calculation formula: D=d−d×n×(arcsin(w/d))/π−2δ; in the calculation formula, D: inner diameter of the sheath catheter after being compressed by the chuck; d: inner diameter of the chuck before compression; n: the number of first slots; w: width of the first slot; δ: thickness of a wall of the sheath catheter;
or D: inner diameter of the sheath catheter being compressed by the chuck; d: inner diameter of the chuck before compression; n: the number of second slots; w: width of the second slot; 8: thickness of a wall of the sheath catheter.

Further, when an outer diameter of the sheath catheter is 4 fr to 11 fr, two to twelve first slots or second slots are provided.

Further, when an outer diameter of the sheath catheter is 9 fr, a width of the first slot or the second slot is 0.18 mm to 0.78 mm.

Further, each of an outer wall of the outer cylinder body and an outer wall of the inner cylinder body is provided with an anti-slip pattern.

In order to solve the above-mentioned technical problem, a second technical solution of the present application provides a medical catheter including a driving shaft and a sheath catheter located outside the driving shaft, and the fixing-locking structure as described above is arranged outside of the sheath catheter.

In order to solve the above-mentioned technical problem, a third technical solution of the present application provides a blood pump including a pump device, a catheter and an extracorporeal driving device, the catheter includes a driving shaft and a sheath catheter located outside the driving shaft, the extracorporeal driving device drives the pump device through the driving shaft, and the fixing-locking structure as described above is arranged outside of the sheath catheter.

The present application has the following beneficial effects compared with a related art: the fixing-locking structure, the medical catheter and the blood pump provided by the present application are provided with the chuck. The clamping part is arranged at each of two ends of the chuck. The end of the chuck abutting against the inner cylinder body is provided with the first clamping part. The first clamping part is provided with a plurality of first slots evenly spaced apart from one another in the circumferential direction. The inner wall of the first end of the inner cylinder body is provided with a first compressing-fitting part that abuts against the first clamping part. The end of the chuck abutting against the outer cylinder body is provided with a second clamping part. The second clamping part is provided with a plurality of second slots evenly spaced apart from one another in the circumferential direction. The inner wall of the outer cylinder body corresponding to the first chamber is provided with a second compressing-fitting part that abuts against the second clamping part. The compressing-fitting parts through which the inner cylinder body and the outer cylinder body respectively abutting against the clamping parts compress the clamping parts, so that the sheath catheter of the catheter can be fixed and locked. Each of the clamping parts is provided with the slot, so that the sheath catheter can be deformed outwards at the slot, the deformation of the sheath catheter can be controlled, and the minimum inner diameter of the sheath catheter after being deformed can be estimated. Each of the clamping parts and the compressing-fitting parts is in the shape of the frustum of the cone. Faces of the frustum abut against each other, so that when locking in the axial direction, a radial pressure can be achieved, and a sufficient static friction can be ensured, so as to ensure the fixing-locking effect and sufficiently ensure the functionality of the driving shaft. Furthermore, the chuck is the cylindrical elastic structure, which means that the chuck is made of an elastic material such as nylon, rubber, polyethylene or a combination of rubber and polyethylene. A hardness of the chuck is harder than that of the sheath catheter. The material of the inner cylinder body and the outer cylinder body may be high-density polyethylene (HDPE), low-density polyethylene (LDPE) or linear low-density polyethylene (LLDPE).

### BRIEF DESCRIPTION OF THE DRAWINGS

FIG. 1 shows a cross-sectional view of a locking structure in a related art during locking;

FIG. 2 shows a cross-sectional view of a locking head structure of another locking structure in a related art;

FIG. 3 shows an exploded view of a fixing-locking structure according to an embodiment of the present application;

FIG. 4 shows an exploded sectional view of a fixing-locking structure according to an embodiment of the present application;

FIG. 5 shows a sectional view of a fixing-locking structure during locking a sheath catheter according to an embodiment of the present application;

FIG. 6 shows a schematic view of a fixing-locking structure during locking a sheath catheter according to an embodiment of the present application;

FIG. 7 shows a sectional view of a fixing-locking structure during locking a sheath catheter according to an embodiment of the present application;

FIG. 8 shows an exploded view of a fixing-locking structure in a snapping connection manner according to an embodiment of the present application;

FIG. 9 shows an exploded sectional view of a fixing-locking structure in a snapping connection manner according to an embodiment of the present application;

FIG. 10 shows a structural schematic view of a medical catheter according to an embodiment of the present application; and

FIG. 11 shows a structural schematic view of a blood pump according to an embodiment of the present application.

In the drawings
1. inner cylinder body; 2. chuck; 3. outer cylinder body; 4. sheath catheter; 5. locking ring; 6. driving shaft; 7. pump device; 8. extracorporeal driving device; 9. locking head; 91. proximal end of locking head; 92. distal end of locking head; 11. first compressing-fitting part; 12. external thread; 13. positioning pin; 21. first clamping part; 22. first slot; 23. second clamping part; 24. second slot; 31. first chamber; 32. second compressing-fitting part; 33. internal thread; 34. ring groove; 35. guiding groove.

### DETAILED DESCRIPTION

The present application will be further described in conjunction with drawings and embodiments.

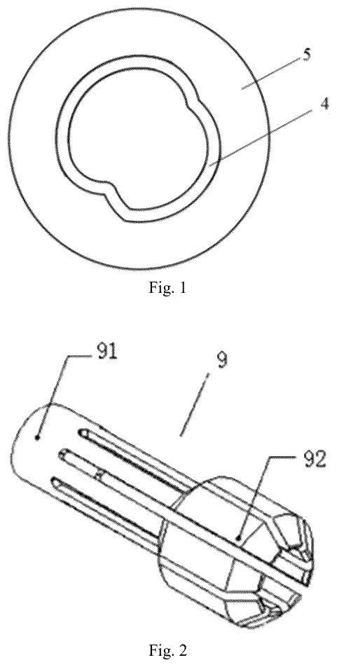

As shown in FIG. 1, a locking structure in a relative art includes a locking cap and a locking base that compress an elastic locking ring 5 in an axial direction when they rotate and lock with each other. According to the principle of volume conservation, the compression in a radial direction can be achieved to achieve the purpose of locking. Although this locking method can achieve the locking, the radial deformation of the elastic locking ring is difficult to be controlled during, and the deformation of a corresponding sheath catheter 4 is difficult to be predicted, when the locking structure is compressed in the radial direction radial. This situation may lead to the following problems: firstly; a locking force is unstable or the consistency of the locking force is poor; when the elastic locking ring 5 is compressed in the axial direction, due to the deformation of a catheter wall of the locking ring 5, which cannot be predicted, the morphologies of contact surfaces between the locking ring 5 and the sheath catheter 4 are different; when the locking cap and the locking base rotate in the same displacement, the locking forces generated at different positions on an outer wall of the locking ring 5 are different; secondly; the internal collapse of the catheter may occur during locking; due to the uncontrolled deformation of the catheter wall of the locking ring 5 during using, an operating force of compressing the elastic locking ring 5 may be too large in order to achieve the required locking force, which may result in the excessive local deformation of the sheath catheter 4, the collapse inside the catheter and other unexpected structural changes; thirdly; the minimum inner diameter of the sheath catheter decreases after locking; during the clinical practice of a percutaneous intervention type blood pump, the catheter at least includes an internal mechanical transmission shaft and the external sheath catheter 4; if the deformation of the inner diameter of the sheath catheter is too much, the mechanical transmission shaft inside the catheter may malfunction.

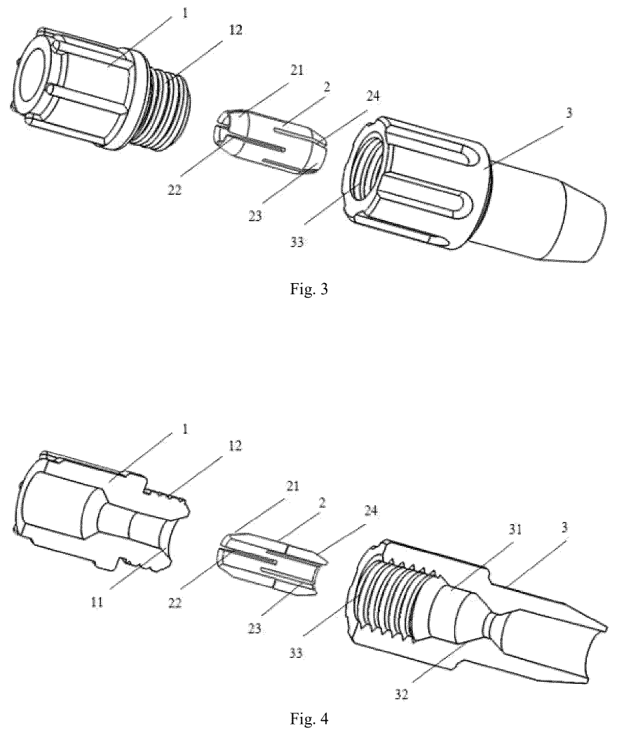

As shown in FIG. 2, a locking structure in another related art includes a locking cap and a connecting part that compress a locking head 9 in an axial direction when they rotate and lock with each other. The locking head 9 sleeve a conveying catheter. The conveying catheter includes a sheath catheter in the exterior, and the locking head 9 is a retractable multi-petal contraction structure. After contracting, the conveying catheter is locked. During a process of being compressed by a force, the deformation of a proximal end 91 of the locking head drives a multi-petal structure of a distal end 92 of the locking head to contract. Therefore, the locking head 9 is designed to be thick at the distal end and thin at the proximal end, and a length of a separation slot of the multi-petal contraction structure, which extends from the distal end to the proximal end, is relatively long, extending from the distal end to the proximal end. The relatively thin proximal end and the relatively long separation slot are prone to deformation to achieve the contraction at the distal end. Each petal of the multi-petal structure of the locking head 9 is a cantilever structure. In order to achieve the locking at the distal end, a larger pressure needs to be exerted at the proximal end, which may result in the stress concentration at the proximal end 91 of the locking head. The stress concentration at the proximal end 91 of the locking head is prone to lead to the fracture and damage. In addition, in order for the distal end of the locking head 92 to achieve the force for locking the sheath catheter of the catheter, the multi-petal structure at the distal end of the locking head 92 needs to contract significantly. The contraction of the multi-petal structure can decrease the inner diameter of the catheter, thereby leading to a larger deformation of the sheath catheter and a smaller space inside the catheter, so that a situation where the mechanical transmission shaft is arranged in the catheter is not suitable for application. As described above, the proximal end is an end close to the physician, and the distal end is an end far away from the physician.

Based on the above problems, the inventors of the present application provide a fixing-locking structure, a medical catheter and a blood pump.

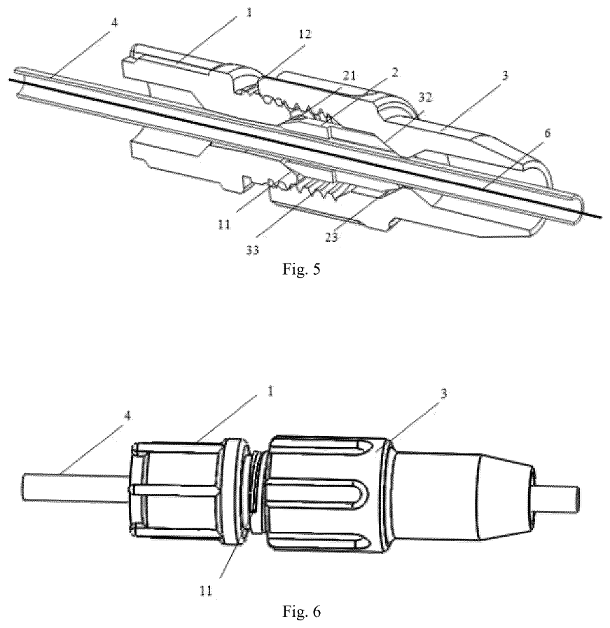

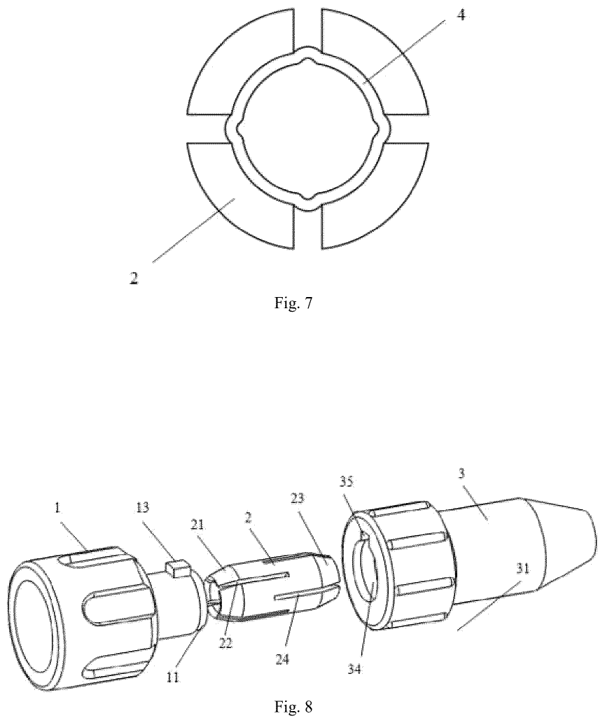

FIG. 3 shows an exploded view of a fixing-locking structure according to an embodiment of the present application; FIG. 4 shows an exploded sectional view of a fixing-locking structure according to an embodiment of the present application; FIG. 5 shows a sectional view of a fixing-locking structure during locking a sheath catheter according to an embodiment of the present application; FIG. 6 shows a schematic view of a fixing-locking structure during locking a sheath catheter according to an embodiment of the present application; and FIG. 7 shows a sectional view of a fixing-locking structure during locking a sheath catheter according to an embodiment of the present application.

Referring to FIG. 3 to FIG. 7, the fixing-locking structure provided by the embodiments of the present application includes an inner cylinder body 1, a chuck 2 and an outer cylinder body 3, that sleeve a catheter. The chuck 2 is in a cylindrical elastic structure. A distal end of the outer cylinder body 3 is provided with a first chamber 31 that accommodates the chuck 2, and a proximal end of the inner cylinder body 1 is connected to the distal end of the outer cylinder body 3. One end of the chuck 2 abuts against the proximal end of the inner cylinder body 1, and the other end abuts against an inner wall of the first chamber 31. Herein, the proximal end of the inner cylinder body is a first end thereof, and the distal end of the outer cylinder body is a first end thereof.

Optionally, in some other embodiments, the positions of the outer cylinder body 3 and the inner cylinder body 1 can be interchanged. In this case, the proximal end of the outer cylinder body 3 is provided with the first chamber 31 that accommodates the chuck 2, and the distal end of the inner cylinder body 1 is connected to the proximal end of the outer cylinder body 3. One end of the chuck 2 abuts against the distal end of the inner cylinder body 1, and the other end abuts against the inner wall of the first chamber 31. Herein, the distal end of the inner cylinder body is a first end thereof, and the proximal end of the outer cylinder body is a first end thereof.

The chuck 2 is the cylindrical elastic structure, which means that the chuck 2 is made of an elastic material such as nylon, rubber, polyethylene or a combination of rubber and polyethylene. A hardness of the chuck 2 is harder than that of the sheath catheter 4. The material of the inner cylinder body 1 and the outer cylinder body 3 may be high-density polyethylene (HDPE), low-density polyethylene (LDPE) or linear low-density polyethylene (LLDPE).

Specifically, each of two ends of the chuck 2 is provided with a clamping part, and the clamping part is provided with a plurality of slots in a circumferential direction. A compressing-fitting part is formed at a position where each of the inner cylinder body 1 and the outer cylinder body 3 abuts against the clamping part. When the proximal end of the inner cylinder body 1 is connected to the distal end of the outer cylinder body 3, the compressing-fitting part compresses the clamping part to contract inwards. The sheath catheter 4 deforms outwards in its radial direction at the slots and is fixed and locked by the chuck 2 at the same time. A deformation position is reserved in advance at the slots, so that a deformation direction of the sheath catheter 4 is in its radial direction outwards. A deformed portion of the sheath catheter 4 is pressed into the slots, and during the clamping part contracting inwards, a static friction force on the sheath catheter 4 gradually increases, so that it can be ensured that when the required maximum static friction is achieved, an inner diameter of the sheath catheter 4 after being deformed is uniform and controllable, and the deformation amount is smaller than that in the related art.

Specifically, the end of the chuck 2 that abuts against the inner cylinder body 1 is provided with a first clamping part 21, which is provided with a plurality of first slots 22 in the circumferential direction. An inner wall of the proximal end of the inner cylinder body 1 is provided with a first compressing-fitting part 11 that abuts against the first clamping part 21. The end of the chuck 2 that abuts against the outer cylinder body 3 is provided with a second clamping part 23, which is provided with a plurality of second slots 24 in the circumferential direction. An inner wall of the proximal end of the first chamber 31 is provided with a second compressing-fitting part 32 that abuts against the second clamping part 23. The two ends of the chuck 2 are symmetrically provided with the clamping parts, respectively; so that the overall forces exerted on the two ends of chuck 2 can be uniform, and the local stress can be reduced. The compressing-fitting part compresses the clamping part to contract inwards. The deformation of the sheath catheter 4 at the clamping part is the largest, and the maximum deformation occurs at a place where the maximum normal stress is exerted, thereby reducing the deformation. Compared with the locking structure in the related art in FIG. 2, the deformation of the proximal end 91 of the locking head drives the multi-petal structure of the distal end 92 of the locking head to contract and lock. In order for the distal end 92 of the locking head to achieve the same locking force as the clamping part of the present application, a larger pressure needs to be exerted to the proximal end 91 of the locking head. The multi-petal structure of the distal end 92 of the locking head requires a larger contraction for the structure of the chuck 2 that is symmetrically locked at the two ends, which can result in the larger deformation of the sheath catheter 4 and the smaller space inside the catheter smaller, so that the situation where the mechanical transmission shaft is arranged in the catheter is not suitable for application. In addition, the locking head 9 has a structure that is thick at the distal end and thin at the proximal end. Due to the thin proximal end, a portion of the proximal end where the stress concentration occurs is prone to fracture and damage. While the chuck 2 with the symmetrical structure bears the uniform stresses at the two ends, so as to bear the smaller local stress, so that there is no need to consider a thickness of a wall. That is, the thickness of the wall of the chuck 2 can be formed uniformly. Compared with the structure of the locking head 9 with the thick distal end and the thin proximal end, the structure of chuck 2 is simpler and easier to be manufactured, so that the production cost can be reduced.

Specifically, each of the first clamping part 21 and the first compressing-fitting part 11 is in a shape of a frustum of a cone, and each of the second clamping part 23 and the second compressing-fitting part 32 is in a shape of a frustum of a cone. Faces of the frustum abut against each other, the first compressing-fitting part 11 and the second compressing-fitting part 32 with the shape of the frustum of the cone can guide the first clamping part 21 and the second clamping part 23 to be contracted inwards in the radial direction of the sheath catheter 4, so that when the first clamping part 21 and the second clamping part 23 of the chuck 2 are locked in the axial direction, they can exert a radial pressure to the sheath catheter 4, so as to generate a sufficient static friction, and produce a fixing-locking effect. A top angle of the cone of the first clamping part 21 is smaller than a top angle of the cone of the first compressing-fitting part 11, and a top angle of the cone of the second clamping part 23 is smaller than a top angle of the cone of the second compressing-fitting part 32, so that the clamping parts are in line contact with the compressing-fitting parts to avoid the problem of a high friction force caused by the face contact.

Specifically, the first slots 22 and the second slots 24 are both on an outer surface of the chuck 2 and extend in the axial direction of the chuck 2. The first slots 22 and the second slots 24 are staggered with each other in the circumferential direction of the chuck 2. That is, in the circumferential direction of the chuck 2, projections of the first slots 22 in the axial direction of the chuck 2 are spaced apart from the projections of the second slots 24 in the axial direction of the chuck 2. In addition, in the circumferential direction of the chuck 2, each of the projections of the first slots 22 in the axial direction of the chuck 2 is adjacent to the projection of another first slot 22 in the axial direction of the chuck 2; or in the circumferential direction of the chuck 2, each of the projections of the second slots 24 in the axial direction of the chuck 2 is adjacent to the projection of another second slot 24 in the axial direction of the chuck 2; or in the circumferential direction of the chuck 2, each of the projections of the first slots 22 in the axial direction of the chuck 2 is adjacent to the projection of the second slot 24 in the axial direction of the chuck 2.

Optionally, the plurality of first slots 22 are formed on the first clamping part 21 in equidistant, and the plurality of second slots 24 are formed on the second clamping part 23 in equidistant. The equidistant arrangement can make the forces more evenly during the locking process.

Optionally, a width of the first slot is equal to a width of the second slot, and the number of first slots is equal to the number of second slots. Thus, the first clamping part 21 and the second clamping part 23 can exert the same pressure to the sheath catheter 4 during the locking process, resulting in the more uniform deformation of the sheath catheter 4 at the two ends of the chuck 2.

In some other embodiments, the first slots 22 and the second slots 24 can be symmetrically arranged and placed relative to the same central axis.

As shown in FIG. 5, the catheter includes a driving shaft 6 and the sheath catheter 4 arranged outside the driving shaft 6. The driving shaft 6 is connected to an extracorporeal driving device to drive a pump device. Therefore, a sufficient space must be reserved inside the sheath catheter to allow the driving shaft 6 to rotate to drive the pump device. Therefore, when the sheath catheter 4 is fixed by the fixing-locking structure, it is necessary to ensure that the space inside the sheath catheter 4 is controllable and predictable. In FIG. 1 of the related art, when the locking ring 5 locks the sheath catheter 4 of the catheter, the deformation of the sheath catheter 4 is uncontrolled, which can easily cause the unexpected structural change such as collapse, so that the space inside sheath catheter 4 is uncontrollable.

It should be noted that the fixing-locking structure in the embodiments of the present application sleeves the catheter, specifically sleeves the sheath catheter of the catheter. Optionally, there is at least one sheath catheter. The number of sheath catheters is not limited. For example, a first sheath catheter and a second sheath catheter can be provided, and the second sheath sleeves the first sheath catheter, the fixing-locking structure sleeves the second sheath catheter, and so on.

Specifically, the driving shaft 6 is a mechanical transmission structure that, optionally, can extend through the entire catheter. Optionally, the driving shaft 6 is composed of a flexible cable or includes a flexible cable, which can be composed of fiber layers with different orientations. Optionally, the driving shaft 6 can be composed of a plurality of coaxial winding sets. The proximal end of driving shaft 6 is connected to the extracorporeal driving device 8, and driving shaft 6 is used to transfer the torque from the extracorporeal driving device 8 to the pump device 7 at the distal end of the driving shaft 6.

The pump device 7 is inserted into the heart or the blood vessel through the catheter and is used to pump blood through a circulatory system. If used in the heart, the pump device can reduce the workload on the heart of the patient, for example, the heart can be recovered after the heart disease. Generally, the pump device 7 includes an impeller arranged in a pump casing. The pump device can be arranged in a fixed or expandable structure. The extracorporeal driving device 8 may be an electric motor or a pneumatic motor. In the fixing-locking structure in the embodiments of the present application, the sheath catheter 4 of the catheter deforms outwards in its radial direction at the slots, resulting in the minimum inner diameter of the sheath catheter 4 of the catheter after being compressed and locked by the chuck 2. Optionally, the width of the first slot is equal to the width of the second slot, and the number of the first slots is equal to the number of the second slots. Thus, the minimum inner diameters of the sheath catheter 4 at two ends of the chuck 2 after being compressed and locked in the chuck 2 can be the same. It can be estimated by the following calculation formula: D=d−d×n×(arcsin(w/d))/π−2δ; in the calculation formula, D: inner diameter of the sheath catheter 4 after being compressed by the chuck 2; d: inner diameter of the chuck 2 before compression; n: the number of first slots; w: width of the first slot; δ: thickness of a wall of the sheath catheter 4; or D: inner diameter of the sheath catheter being compressed by the chuck; d: inner diameter of the chuck before compression; n: the number of second slots; w: width of the second slot; δ: thickness of a wall of the sheath catheter. Thus, the minimum inner diameter of the sheath catheter 4 after being compressed, locked and fixed can be calculated by the number of slots and the width of slot, thereby ensuring the internal size of the sheath catheter 4. In order to ensure the uniform normal stress exerted on the sheath catheter 4 by the fixing-locking structure, the cumulative width of the plurality of slots, i.e. a value of the width of the slot multiplied by the number of slots (the product of the width of the slot and the number of slots), should be minimized as much as possible. In the case of a certain cumulative width of the slots, optionally, the number of slots may be increase and the width of the slot may be reduced, so that the deformation of the sheath catheter 4 can be more uniform. However, due to a generally small outer diameter size of an interventional catheter instrument, too many number of slots may increase the processing difficulty and cost. In addition, the cumulative width of the slots should be determined by the deformation of the material of the corresponding sheath catheter 4 under the required maximum static friction force to determine the minimum value of the cumulative width.

In FIG. 1 of the related art, since the maximum static friction force is determined by the pressure and the friction coefficient, when the material of the sheath catheter 4 is extremely soft, the sheath catheter 4 will deform with the contraction of the locking ring 5, so that the locking ring 5 is difficult to achieve the required maximum static friction force by reducing the limited size. In the embodiments of the present application, the chuck 2 can exert the normal stress to the sheath catheter 4 by contracting the slots, thereby clamping the sheath catheter 4 and obtaining the required maximum static friction force by contracting a relatively small size of the sheath catheter 4.

In the fixing-locking structure, the number of slots in the clamping part at the end of the chuck 2 can be set to 2 to 16, and distributed evenly. When the diameter of the sheath catheter 4 is small, such as 9 fr (French, unit of circumference measurement) or smaller, due to a relatively small contact area between the chuck 2 and the sheath catheter 4, the number of slots should be appropriately reduced to prevent the stress concentration; generally, 2 to 8 are relatively appropriate. Optionally, when the diameter of the sheath catheter 4 is 9 fr, the corresponding width of the slot ranges from 0.78 mm (2 slots) to 0.18 mm (8 slots); when the size of the sheath catheter 4 is 4 fr to 11 fr, the number of slots in the clamping part at the end of the chuck 2 is 2 to 12; when the size of the sheath catheter 4 is particularly large (generally larger than 11 fr), the number of slots can be appropriately increased according to the actual situation to avoid the concentrated deformation positions and unexpected deformation.

Referring to FIG. 3 to FIG. 7, in the fixing-locking structure in the embodiments of the present application, a connection structure between the inner cylinder body 1 and the outer cylinder body 3 is a threaded connection structure. The threaded connection structure includes an external thread 12 arranged on an outer wall of the proximal end of the inner cylinder body 1 and an internal thread 33 arranged on an inner wall of the distal end of the outer cylinder body 3, and the external thread 12 matches with the internal thread 33. When the proximal end of the inner cylinder body 1 gradually rotates into the distal end of the outer cylinder body 3, the compressing-fitting part gradually compresses the clamping part to contract it inwards, and the sheath catheter 4 of the catheter deforms outwards at the slots.

In some other embodiments, optionally; the position of the inner cylinder body 1 and the position of the outer cylinder body 3 can be interchanged. The threaded connection structure includes an external thread 12 arranged on an outer wall of the distal end of the inner cylinder body 1 and an internal thread 33 arranged on an inner wall of the proximal end of the outer cylinder body 3, and the external thread 12 matches with the internal thread 33. When the distal end of the inner cylinder body 1 gradually rotates into the proximal end of the outer cylinder body 3, the compressing-fitting part gradually compresses the clamping part to contract it inwards, and the sheath catheter 4 of the catheter deforms outwards at the slots.

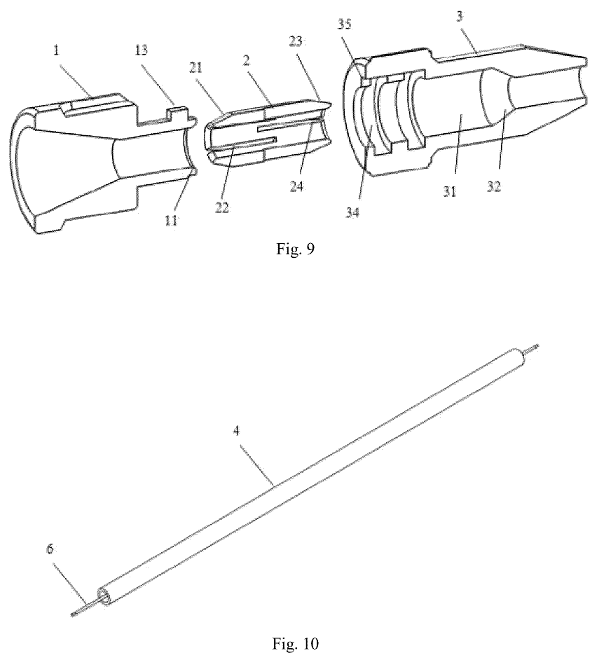

Referring to FIG. 8 and FIG. 9, in the fixing-locking structure in the embodiments of the present application, the connection structure between the inner cylinder body 1 and the outer cylinder body 3 is a snapping connection structure. The snapping connection structure includes a positioning pin 13 arranged on the outer wall of the proximal end of the inner cylinder body 1 and a ring groove 34 formed on the inner wall of the distal end of the outer cylinder body 3. The ring groove 34 is formed on the inner wall of the outer cylinder body 3 and arranged in the circumferential direction. There is at least one ring groove 34. A guiding groove 35 is formed on the inner wall of the distal end of the outer cylinder body 3 and arranged in the axial direction. The guiding groove 35 communicates with the ring groove 34. The positioning pin 13 passes through the guiding groove 35 in the axial direction, slidably moves into the ring groove 34 and rotates to be fixed into the ring groove 34. When the inner cylinder 1 is clamped into the ring groove 34 of the outer cylinder body 3 through the positioning pin 13, the compressing-fitting part compresses the clamping part to contract inwards. The sheath catheter 4 of the catheter deforms outwards in its radial direction at the slots and is fixed and locked. A deformation space is provided in the slots in advance, so that the deformation direction of the sheath catheter 4 is in its radial direction outwards. A deformed portion of the sheath catheter 4 is pressed into the slots. During the process of the clamping part contracting inwards, the static friction force of the sheath catheter 4 gradually increases, so that it can be ensured that when the required maximum static friction is achieved, an inner diameter of the sheath catheter 4 after being deformed can be uniform and controllable, and the minimum inner diameter of the sheath catheter is large.

In some other embodiments, optionally; the snapping connection structure includes a ring groove 34 formed on the outer wall of the proximal end of the inner cylinder body 1 and a positioning pin 13 arranged on the inner wall of the distal end of the outer cylinder body 3. The ring groove 34 is formed in the circumferential direction of the outer wall of the inner cylinder body 1. There is at least one ring groove 34. A guiding groove 35 is formed in the axial direction of the inner wall of the distal end of the outer cylinder body 3. The positioning pin 13 passes through the guiding groove 35 in the axial direction, slidably moves into the ring groove 34 and rotates to be fixed into the ring groove 34. When the inner cylinder 1 is connected to the positioning pin 13 of the outer cylinder body 3 through the ring groove 34, the compressing-fitting part compresses the clamping part to contract inwards. The sheath catheter 4 of the catheter deforms outwards in its radial direction at the slots and is fixed and locked. The deformation position is left at the slots in advance, so that the deformation direction of the sheath catheter 4 is in its radial direction outwards. A deformed portion of the sheath catheter 4 is pressed into the slots. During the process of the clamping part contracting inwards, the static friction force of the sheath catheter 4 gradually increases, so that it can be ensured that when the required maximum static friction is achieved, an inner diameter of the sheath catheter 4 after being deformed can be uniform and controllable, and the minimum inner diameter of the sheath catheter is large.

Optionally, each of an outer wall of the outer cylinder body 3 and an outer wall of the inner cylinder body 1 is provided with an anti-slip pattern for easy holding and locking.

In the embodiments of the present application, when the fixing-locking structure is used in a percutaneous interventional blood pump, the inner cylinder body 1 is connected to a suture system, the chuck 2 sleeves the sheath catheter 4, and the outer cylinder body 3 is connected to an antibacterial sleeve structure. In the actual surgical process, for different patients, relative positions of the suture system on the sheath catheter 4 are different, so that the suture system is required to be movable freely on the sheath catheter 4. In an initial state, the inner cylinder body 1 is not connected to the outer cylinder body 3. When the suture system reaches a designated position with the inner cylinder body 1, the first clamping part of the chuck 2 abuts against the inner cylinder body 1, the second clamping part of the chuck 2 abuts against the outer cylinder body 3, and the inner cylinder body 1 is connected to the outer cylinder body 3. The connection between the inner cylinder body 1 and the outer cylinder body 3 ensures that the suture system is tightly fastened to the antibacterial sleeve structure. In addition, when the inner cylinder body 1 is connected to the outer cylinder body 3, the compressing-fitting part compresses the clamping part of the chuck 2. The clamping part of the chuck 2 contracts inwards through the slots and is fixed to the sheath catheter 4, thereby fixing the suture system. The sheath catheter 4 deforms uniformly outwards in its radial direction at the slots, so as to avoid the excessive deformation of sheath catheter 4.

Referring to FIG. 10, the embodiments of the present application provides a medical catheter, including a driving shaft 6 and a sheath catheter 4 located outside the driving shaft 6. A fixing-locking structure is arranged outside of the sheath catheter 4 to fix the sheath catheter 4.

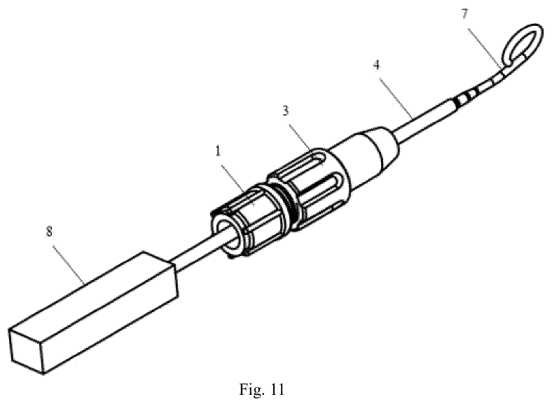

Referring to FIG. 11, the embodiments of the present application provides a blood pump including a pump device 7, a catheter and an extracorporeal driving device 8. The catheter includes a driving shaft 6 and a sheath catheter 4 located outside the driving shaft 6. The extracorporeal driving device 8 drives the pump device 7 through the driving shaft 6, and a fixing-locking structure is arranged outside of the sheath catheter 4 to fix the sheath catheter 4.

In summary, the fixing-locking structure, the medical catheter and the blood pump provided by the embodiments of the present application are provided with the chuck 2 including the clamping part, and the sheath catheter 4 of the catheter is fixed and locked by the compressing-fitting parts abutting against the clamping parts through the inner cylinder body 1 and outer cylinder body 3, respectively. Each of the clamping parts is uniformly provided with the slots in the circumferential direction, so that the sheath catheter 4 is deformed outwards in its radical direction at the slots. The deformation of sheath catheter 4 is controllable, and the minimum inner diameter of the sheath catheter 4 after being deformed can be estimated. Each of the clamping parts and the compressing-fitting parts is in the shape of the frustum of the cone. Faces of the frustum abut against each other, so that when locking in the axial direction, the radial pressure can be achieved, the sufficient static friction can be ensured, and the fixing-locking effect can be ensured.

Although the present application has been disclosed according to the preferred embodiments as described above, the preferred embodiments are not intended to limit the present application. Any skilled in the art may make slight modifications and improvements within the gist and scope of the present application. Therefore, the scope of the present application shall be subject to the claims.

1. A fixing-locking structure comprising an inner cylinder body, a chuck and an outer cylinder body that sleeve a catheter, and the chuck being in a cylindrical shape;
a first end of the outer cylinder body being provided with a first chamber to accommodate the chuck, a first end of the inner cylinder body being connected to the first end of the outer cylinder body, one end of the chuck abutting against an inner wall of the first end of the inner cylinder body, and the other end of the chuck abutting against an inner wall of the outer cylinder body corresponding to the first chamber;
the end of the chuck abutting against the inner cylinder body being provided with a first clamping part, the inner wall of the first end of the inner cylinder body being provided with a first compressing-fitting part that abuts against the first clamping part, the end of the chuck abutting against the outer cylinder body being provided with a second clamping part, and the inner wall of the outer cylinder body corresponding to the first chamber being provided with a second compressing-fitting part that abuts against the second clamping part; when the first end of the inner cylinder body is connected to the first end of the outer cylinder body, the first compressing-fitting part compressing the first clamping part to contract the first clamping part inwards, and the second compressing-fitting part compressing the second clamping part to contract the second clamping part inwards.

2. The fixing-locking structure according to claim 1, wherein the chuck is formed as an elastic body.

3. The fixing-locking structure according to claim 1, wherein the first clamping part is provided with a first slot in a circumferential direction, the second clamping part is provided with a second slot in the circumferential direction, both of the first slot and the second slot are formed on an outer peripheral face of the chuck and extend in an axial direction of the chuck, and the first slot and the second slot are staggered with each other in the circumferential direction of the chuck.

4. The fixing-locking structure according to claim 3, wherein at least two first slots are formed on the chuck and evenly spaced apart from one another, and at least two second slots are formed on the chuck and evenly spaced apart from one another.

5. The fixing-locking structure according to claim 1, wherein each of the first clamping part, the second clamping part, the first compressing-fitting part and the second compressing-fitting part is in a shape of a frustum of a cone, a top angle of the cone of the first clamping part is smaller than a top angle of the cone of the first compressing-fitting part, and a top angle of the cone of the second clamping part is smaller than a top angle of the cone of the second compressing-fitting part.

6. The fixing-locking structure according to claim 1, wherein the first end of the inner cylinder body is connected to the first end of the outer cylinder body through a threaded connection structure, the threaded connection structure comprises an external thread arranged on an outer wall of the first end of the inner cylinder body and an internal thread arranged on an inner wall of the first end of the outer cylinder body, and the external thread matches with the internal thread.

7. The fixing-locking structure according to claim 1, wherein the first end of the inner cylinder body is connected to the first end of the outer cylinder body through a snapping connection structure, the snapping connection structure comprises a positioning pin arranged on an outer wall of the first end of the inner cylinder body and a ring groove formed on an inner wall of the first end of the outer cylinder body, the ring groove is formed on the inner wall of the outer cylinder body in a circumferential direction, a guiding groove is formed on the inner wall of the first end of the outer cylinder body in an axial direction, the guiding groove communicates with the ring groove, the positioning pin passes through the guiding groove in the axial direction, slidably moves into the ring groove and rotates to be fixed into the ring groove, and the inner cylinder body is fixed into the ring groove of the outer cylinder body through the positioning pin.

8. The fixing-locking structure according to claim 3, wherein a minimum inner diameter of a sheath catheter of the catheter after being compressed, locked and fixed by the chuck is estimated by a following calculation formula:
$$D = d - d \times n \times ( arcsin ( w / d ) ) / π - 2 δ ;$$
in the calculation formula, D: inner diameter of the sheath catheter after being compressed by the chuck; d: inner diameter of the chuck before compression; n: the number of first slots; w: width of the first slot; δ: thickness of a wall of the sheath catheter; or
D: inner diameter of the sheath catheter being compressed by the chuck; d: inner diameter of the chuck before compression; n: the number of second slots; w: width of the second slot; δ: thickness of a wall of the sheath catheter.

9. The fixing-locking structure according to claim 3, wherein when an outer diameter of the sheath catheter is 4 fr to 14 fr, two to twelve first slots or second slots are provided.

10. The fixing-locking structure according to claim 3, wherein when an outer diameter of the sheath catheter is 9 fr, a width of the first slot or the second slot is 0.18 mm to 0.78 mm.

11. The fixing-locking structure according to claim 1 wherein each of an outer wall of the outer cylinder body and an outer wall of the inner cylinder body is provided with an anti-slip pattern.

12. A medical catheter comprising a driving shaft and a sheath catheter located outside the driving shaft, and the fixing-locking structure according to claim 1 being arranged outside of the sheath catheter.

13. A blood pump comprising a pump device, a catheter and an extracorporeal driving device, the catheter comprising a driving shaft and a sheath catheter located outside the driving shaft, the extracorporeal driving device driving the pump device through the driving shaft, and the fixing-locking structure according to claim 1 being arranged outside of the sheath catheter.
